(19) United States
(12) Patent Application Publication
(10) Pub. No.: US 2022/0214326 A1
(43) Pub. Date: Jul. 7, 2022

(54) BIOPOLYMER ANALYSIS DEVICE, BIOPOLYMER ANALYSIS EQUIPMENT, AND BIOPOLYMER ANALYSIS METHOD

(71) Applicant: HITACHI HIGH-TECH CORPORATION, Tokyo (JP)

(72) Inventors: Yusuke GOTO, Tokyo (JP); Michiru FUJIOKA, Tokyo (JP); Tatsuo NAKAGAWA, Tokyo (JP); Yoshimitsu YANAGAWA, Tokyo (JP); Naoshi ITABASHI, Tokyo (JP)

(73) Assignee: HITACHI HIGH-TECH CORPORATION, Tokyo (JP)

(21) Appl. No.: 17/604,881

(22) PCT Filed: Apr. 24, 2019
(86) PCT No.: PCT/JP2019/017336

§ 371 Date: Oct. 19, 2021

(51) Int. Cl. G01N 33/487; B01L 3/00; C12Q 1/6806

(52) U.S. Cl. CPC G01N 33/48721; B01L 3/502715; B01L 2300/069; B01L 2300/0645; B01L 2400/0421; C12Q 1/6806

(57) ABSTRACT

A biopolymer analysis device includes an insulating thin film that is made of an inorganic material, a first liquid tank and a second liquid tank that are separated by the thin film, a plurality of first electrodes that is arranged in the first liquid tank, and a second electrode that is disposed in the second liquid tank. A water-repellent liquid and a plurality of liquid droplets are introduced into the first liquid tank, the plurality of first electrodes is configured to be able to convey the plurality of droplets introduced into the first liquid tank by electro wetting on dielectric by applying a certain voltage, and the plurality of droplets is conveyed to portions coming into contact with the plurality of first electrodes, and is insulated from each other by the water-repellent liquid.

### TECHNICAL FIELD

The present disclosure relates to a biopolymer analysis device, biopolymer analysis equipment, and a biopolymer analysis method.

### BACKGROUND ART

In a nanopore device, a pore having a diameter of several A to several nm (hereinafter, referred to as a nanopore) is provided in a thin film having a thickness of several A to several tens of nm, an electrolyte solution is brought into contact with both sides of the thin film and a potential difference is generated between both ends of the thin film. Thus, the electrolyte solution can pass through the nanopore. At this time, when an object to be measured in the electrolyte solution passes through the nanopore, since electrical characteristics, particularly a resistance value, of a peripheral portion of the nanopore change, the object to be measured can be detected by detecting the change in the electrical characteristics. When the object to be measured is a biopolymer, the electrical characteristics of the peripheral portion of the nanopore changes in a pattern shape according to a monomer sequence pattern of the biopolymer. In recent years, a method for analyzing a monomer sequence of a biopolymer by using such a change has been actively studied.

Among these studies, the analysis of the monomer sequence based on the principle that the amount of change in ion current observed when the biopolymer passes through the nanopore varies depending on monomer species has been expected. Since the analysis accuracy of the monomer sequence is decided by the amount of change in the ion current, it is desirable that a difference in the amount of ion current between the monomers is large. Such an analysis method can directly read the biopolymer without requiring a chemical operation involving fragmentation of the biopolymer unlike the method in the related art. The nanopore device is used as a DNA base sequence analysis system (DNA sequencer) when the biopolymer is DNA, and is used as an amino acid sequence analysis system (amino acid sequencer) when the biopolymer is protein. These systems are expected as systems capable of decoding a sequence length much longer than in the related art.

In particular, research and development to put the nanopore into practical use as the DNA sequencer by using a blockade current system is active. The blockade current is a decrease amount of the ion current due to a decrease in an effective cross-sectional area through which ions can pass since the biopolymer blocks the nanopore when the biopolymer passes through the nanopore.

As the nanopore device, there are two types of nanopore devices of a bio-nanopore using a protein having a pore at a center embedded in a lipid bilayer membrane and a solid-state nanopore in which a pore is processed in an insulating thin film formed by a semiconductor processing process. In the bio-nanopore, the amount of change in ion current is measured by using a pore (diameter 1.2 nm and thickness 0.6 nm) of a modified protein (such as Mycobacterium smegmatis porin A (MspA)) embedded in the lipid bilayer membrane as a biopolymer detection unit.

On the other hand, in the solid-state nanopore, a structure in which a nanopore is formed in a thin film of silicon nitride (SiN) which is a semiconductor material or a thin film including a monolayer such as a graphene or molybdenum disulfide is used as the nanopore device. In a biopolymer analysis method using the solid-state nanopore, there have been reported so far on measurement of the amounts of blockade currents of an adenine base, a cytosine base, a thymine base, and a guanine base of a homopolymer (NPL 1 and NPL 2).

In the measurement using the nanopore device, there are the following three problems. A first problem is that individual independent channels need to be insulated from each other without leakage of a current between the individual independent channels in order to realize an integrated nanopore device having arrayed parallel channels. When the individual independent channels are not insulated, the individual independent channels interfere with each other, and accurate measurement cannot be performed. Thus, it becomes difficult to perform independent measurement of each channel.

As a second problem, when a sample is depleted during measurement and a measurement throughput is reduced, or when it is desired to measure another sample after a certain sample is sufficiently measured, an effective continuous measurement time needs to be extended by performing smooth sample supply or sample replacement.

As a third problem, when a biomolecule represented by DNA is measured, since a sample collected from a living body is valuable and it is desirable to collect only a small volume, it is necessary to perform measurement even with a small solution volume (small DNA input amount).

In PTL 1, in order to realize an integrated nanopore device using a lipid bilayer membrane and a bio-nanopore, the following method has been attempted. A water-repellent liquid (oil) and an aqueous solution containing a material constituting a lipid bilayer membrane are alternately poured into a resin flow cell having a plurality of parallel wells, and thus, individual droplet portions are spontaneously formed at a bottom portion of each parallel well, and a common solution portion is spontaneously formed at a well ceiling portion. The integration is realized by spontaneously forming the lipid bilayer membrane at an interface between each individual droplet portion and the common solution portion and electrically embedding the bio-nanopore in the membrane.

Unlike the lipid bilayer membrane using self-assembly of the bio-nanopore, in the solid-state nanopore device, since a solid inorganic thin film made of an inorganic material in advance is used, the method as in PTL 1 cannot be applied, and another approach is required to realize the integration. In NPL 3, a method for forming five parallel channels by dividing one inorganic thin film into separate sections by using a microchannel has been attempted.

In NPL 4, a method for realizing parallelization by combining an O-ring of insulating rubber and a resin flow cell for a device having 16 independent thin films has been attempted.

In PTL 2, in order to realize a parallelized solid-state nanopore device having a high degree of integration, a method for using a water-repellent liquid (oil) as an insulator between independent channels has been attempted. Such a water-repellent liquid is realized by a liquid feeding mechanism using a channel. PTL 3 describes a method for providing an insulating film such as a photosensitive resin as an insulating partition wall between independent channels. Such an insulating film is realized by a liquid feeding mechanism using a pressure bonding method.

As described above, what is common to the integrated nanopore devices is that, a common solution tank is provided on one side of the thin film, and a plurality of independent individual solution tanks is provided on the other side. Such a configuration is a basic structure in the integrated nanopore device.

### CITATION LIST

### Patent Literature

PTL 1: WO2014/064443A
PTL 2: JP 6062569 B
PTL 3: JP 2018-96688 A

### Non-Patent Literature

NPL 1: Feng J., et al., Identification of single nucleotides in MoS₂ nanopores. Nat. Nanotechnol. 10, 1070-1076 (2015).
NPL 2: Goto Y., et al., Identification of four single-stranded DNA homopolymers with a solid-state nanopore in alkaline CsCl solution. Nanoscale 10, 20844-20850 (2018).
NPL 3: Tahvildari R., et al., Integrating nanopore sensors within microfluidic channel arrays using controlled breakdown. Lab on a Chip 15, 1407-1411 (2015).
NPL 4: Yanagi I., et al, Multichannel detection of ionic currents through two nanopores fabricated on integrated Si₃N₄ membranes. Lab on a Chip 16, 3340-3350 (2016).

### SUMMARY OF INVENTION

### Technical Problem

However, in the integrated solid-state nanopore system of the related art, it is difficult to achieve both collective injection of solutions into a plurality of independent individual solution tanks and solution (sample) replacement in the individual solution tanks while maintaining insulation between the channels. Although it is easy to replace the solutions by using a channel such as a flow cell, a special jig or a liquid feeding device such as a pump is required to collectively inject the solutions into the individual solution tank, and the device becomes complicated. This problem is remarkable when the degree of integration increases and the channel is minute.

Since the individual solution tank formed by the pressure bonding method as in PTL 3 is a closed space, it is difficult to replace the solutions in the first place.

In the method of the related art, since a solution volume larger than a solution volume of the individual solution tank is required to arrange the solutions in the individual solution tanks, there is also a problem that it is difficult to measure the sample with a small solution volume.

Therefore, the present disclosure provides a technology for achieving both automatic collective injection of solutions into a plurality of individual solution tanks and automatic replacement of the solutions in the individual solution tanks while maintaining insulation between parallel channels.

### Solution to Problem

In order to solve the above problems, a biopolymer analysis device of the present disclosure includes an insulating thin film that is made of an inorganic material, a first liquid tank and a second liquid tank that are separated by the thin film, a plurality of first electrodes that is arranged in the first liquid tank, and a second electrode that is disposed in the second liquid tank. A water-repellent liquid and a plurality of liquid droplets are introduced into the first liquid tank, the plurality of first electrodes is configured to be able to convey the plurality of droplets introduced into the first liquid tank by electro wetting on dielectric by applying a certain voltage, and the plurality of droplets is conveyed to portions coming into contact with the plurality of first electrodes, and is insulated from each other by the water-repellent liquid.

Further features related to the present disclosure will be apparent from the description of the present specification and the accompanying drawings. The aspects of the present disclosure are achieved and realized by elements, combinations of various elements, the following detailed description, and aspects of the appended claims.

The description of the present specification is merely a typical example, and does not limit the scope of claims or application examples of the present disclosure in any sense.

### Advantageous Effects of Invention

According to the present disclosure, it is possible to achieve both automatic collective injection of solutions into a plurality of individual solution tanks and automatic replacement of the solutions in the individual solution tanks while maintaining insulation between parallel channels.

Other objects, configurations, and effects will be made apparent from the following descriptions.

### BRIEF DESCRIPTION OF DRAWINGS

FIG. 1 is a schematic diagram illustrating a biopolymer analysis device with a single channel according to a reference example.

FIG. 2 is a schematic diagram illustrating a biopolymer analysis device with parallel channels according to a reference example.

FIG. 3A is a schematic diagram illustrating a biopolymer analysis device according to a first embodiment.

FIG. 3B is a schematic diagram illustrating the biopolymer analysis device after a nanopore is opened.

FIG. 4 is a schematic diagram illustrating another biopolymer analysis device according to the first embodiment.

FIG. 5 is a schematic diagram illustrating another biopolymer analysis device according to the first embodiment.

FIG. 6 is a flowchart illustrating a biopolymer analysis method according to the first embodiment.

FIG. 7 is a schematic diagram illustrating a biopolymer analysis device according to a second embodiment.

FIG. 8A is a top view of a first liquid tank of the biopolymer analysis device according to the second embodiment.

FIG. 8B is a top view illustrating a scene in which droplets are conveyed.

FIG. 8C is a top view illustrating a state in which all the droplets are arranged at desired positions.

FIG. 9 is a schematic diagram illustrating a biopolymer analysis device according to a third embodiment.

FIG. 10A is a schematic diagram illustrating a state in which a water-repellent liquid remains on a surface of a thin film.

FIG. 10B is a schematic diagram illustrating a structure of a sacrificial layer according to a fourth embodiment.

FIG. 10C is a schematic diagram illustrating another biopolymer analysis device according to the fourth embodiment.

FIG. 11 is a schematic diagram illustrating a biopolymer analysis device according to a fifth embodiment.

FIG. 12 is a schematic diagram illustrating another biopolymer analysis device according to the fifth embodiment.

FIG. 13 is a schematic diagram illustrating a biopolymer analysis device according to a sixth embodiment.

FIG. 14A is a schematic diagram illustrating a biopolymer analysis device according to a seventh embodiment.

FIG. 14B is a schematic diagram illustrating the biopolymer analysis device according to the seventh embodiment.

FIG. 15 is a schematic diagram illustrating biopolymer analysis equipment according to an eighth embodiment.

### DESCRIPTION OF EMBODIMENTS

Hereinafter, embodiments of the present disclosure will be described with reference to the drawings. Although the accompanying drawings illustrate specific embodiments based on the principles of the present disclosure, the drawings are provided for understanding the present disclosure, and are not used for restrictively interpreting the present disclosure.

Biopolymer analysis devices have different configurations depending on a method for introducing biopolymers into a nanopore. In the present specification, a method for introducing biopolymers into a nanopore by electrophoresis will be described as an example. Here, the biopolymer refers to DNA or RNA having a nucleic acid as a monomer, or a protein or a polypeptide having an amino acid as a monomer.

### REFERENCE EXAMPLES

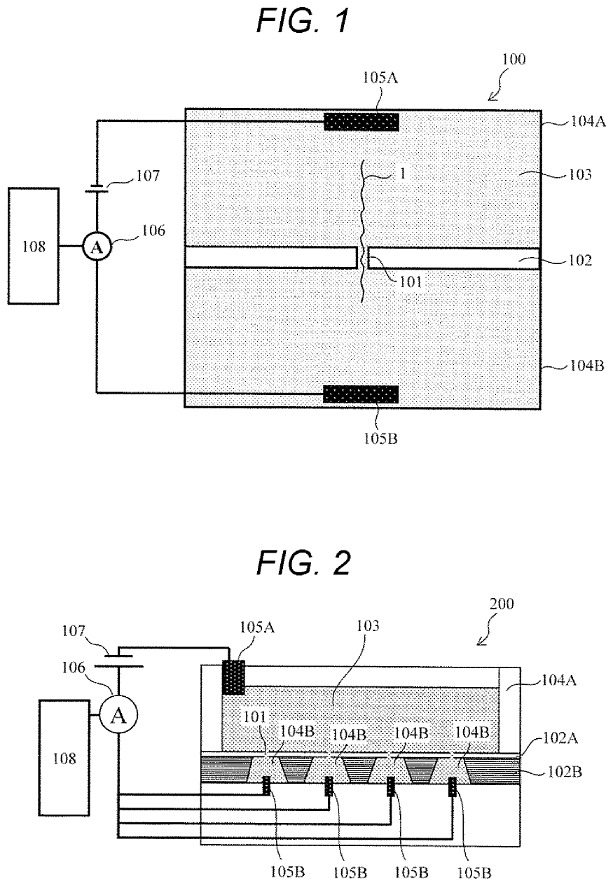

FIG. 1 is a schematic diagram illustrating a biopolymer analysis device 100 having a single nanopore channel according to a reference example. As illustrated in FIG. 1, the biopolymer analysis device 100 includes a thin film 102 having a nanopore 101, a first liquid tank 104A and a second liquid tank 104B storing an electrolyte solution 103, and electrodes 105A and 105B.

The electrodes 105A and 105B are connected to an ammeter 106 and a power supply 107. A voltage is applied to the electrode 105A and the electrode 105B by the power supply 107. The application of the voltage by the power supply 107 is controlled by a computer 108.

The ammeter 106 measures an ion current (blockade current) flowing between the electrode 105A and the electrode 105B. Although not illustrated, the ammeter 106 includes an amplifier that amplifies the current flowing between the electrodes 105A and 105B and an analog-to-digital converter. The ammeter 106 is connected to the computer 108, and the analog-to-digital converter outputs, as a digital signal, a value of the detected ion current to the computer 108.

The computer 108 acquires monomer sequence information of biopolymers 1 based on the value of the ion current (blockade current).

FIG. 2 is a schematic diagram illustrating a biopolymer analysis device 200 as an array device having parallel nanopore channels according to a reference example. The array device refers to a device including a plurality of individual solution tanks partitioned by partition walls. As illustrated in FIG. 2, the biopolymer analysis device 200 is different from the biopolymer analysis device 100 of FIG. 1 in that a plurality of second liquid tanks 104B electrically insulated by a tapered layer 102B as a partition wall is provided, and electrodes 105B are provided in the plurality of second liquid tanks 104B, respectively.

As described above, the first liquid tank 104A is a common solution tank, the second liquid tanks 104B are a plurality of individual solution tanks, and a plurality of independent channels is formed. The electrode 105A is a common electrode, and the electrodes 105B are individual electrodes.

### First Embodiment

<Configuration Example of Biopolymer Analysis Device>

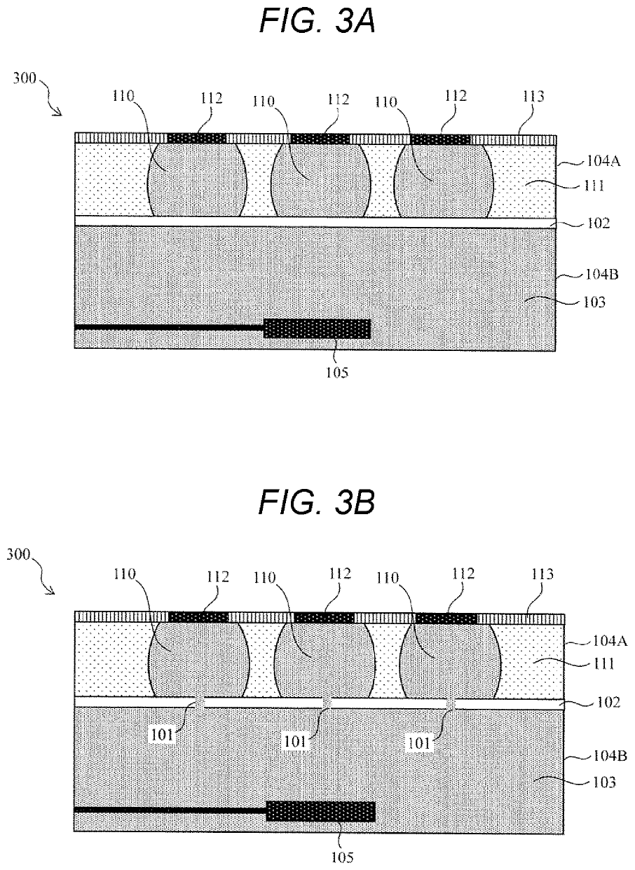

FIG. 3A is a schematic diagram illustrating a biopolymer analysis device 300 according to a first embodiment. As illustrated in FIG. 3A, the biopolymer analysis device 300 is a solid-state nanopore device, and includes a thin film 102 made of an inorganic material, a first liquid tank 104A, a second liquid tank 104B, a common electrode 105 (second electrode), and a substrate 113 having a plurality of individual electrodes 112 (a plurality of first electrodes).

The material of the thin film 102 is an insulating inorganic material that can be formed by a semiconductor microfabrication technique. Examples of the material of the thin film 102 include silicon nitride (SiN), silicon oxide (SiO₂), silicon oxynitride (SiON), hafnium oxide (HfO₂), molybdenum disulfide (MoS₂), a graphene, and the like. A thickness of the thin film 102 can be, for example, from 1 Å to 200 nm, optionally from 1 Å to 100 nm or from 1 Å to 50 nm, for example about 5 nm.

Although not illustrated, the common electrode 105 can be connected to the ammeter 106, the power supply 107, and the computer 108 (controller) illustrated in FIGS. 1 and 2 via wirings, and the plurality of individual electrodes 112 can be connected to the ammeter 106, the power supply 107, and the computer 108 via wirings inside the substrate 113.

As will be described later, the computer 108 controls application of a voltage to the plurality of individual electrodes 112 and the common electrode 105 by the power supply 107. The computer 108 applies a voltage between the plurality of individual electrodes 112 or between each individual electrode 112 and the common electrode 105, and determines positions of droplets 110, whether or not a leak occurs between the droplets 110, or whether or not nanopores are formed in the thin film 102 based on electrical characteristics such as a measured current value. The computer 108 includes a storage (not illustrated), and stores the measured current value or the determination result in the storage.

The plurality of individual electrodes 112 is embedded in the substrate 113. The substrate 113 constitutes a part of the first liquid tank 104A. The material of the substrate 113 may be any material as long as circuit wirings can be mounted, and for example, a PWB substrate or a PCB substrate such as glass epoxy resin is used. Alternatively, the substrate 113 may be a transparent substrate such as a glass substrate.

A plurality of droplets 110 and a water-repellent liquid 111 are introduced into the first liquid tank 104A. Each droplet 110 is electrically insulated from the adjacent droplet 110 by the water-repellent liquid 111, and the droplets are independent of each other. The plurality of droplets 110 comes into contact with the individual electrode 112, respectively, and thus, an electrical operation such as application of a voltage can be performed on each droplet 110.

A certain voltage is applied between the adjacent individual electrodes 112, and thus, the individual electrodes 112 convey the droplets 110 to desired positions by electro wetting on dielectric (EWOD). FIG. 3A illustrates a state in which the droplets 110 are conveyed to positions coming into contact with the individual electrodes 112, and the droplets 110 are separated from each other by the water-repellent liquid 111 and are insulated from each other. Accordingly, the plurality of individual solution tanks (the plurality of channels) is formed.

Application of an EWOD conveying voltage (certain voltage) for operating the individual electrodes 112 as EWOD electrodes is controlled by the computer 108. The EWOD conveying voltage can be set to, for example, 0 to 100V, and is typically set in a range of 10 to 50V. This voltage value changes every time depending on a diameter and a viscosity of the droplet 110, a contact angle between the droplet 110, the water-repellent liquid 111, and the individual electrode 112, an electrode size, or the like, and thus, the voltage value is appropriately adjusted.

The individual electrode 112 is also used to open the nanopores 101 or measure the ion current by applying a voltage between the individual electrode 112 and the common electrode 105.

An electrolyte solution 103 as a common solution is introduced into the second liquid tank 104B. The common electrode 105 is disposed so as to come into contact with the electrolyte solution 103. Here, the plurality of droplets 110 and the electrolyte solution 103 are aqueous solutions containing an electrolyte, and may contain biopolymers to be analyzed.

The volume of the electrolyte solutions 103 can be on the order of microliters or milliliters. The volume of the droplets 110 can be on the order of nanoliter or microliter.

The first liquid tank 104A and the second liquid tank 104B that store a measurement solution coming into contact with the thin film 102 can be appropriately provided with a material, a shape, and a size that do not affect the measurement of the ion current.

The materials of the individual electrode 112 and the common electrode 105 can be materials capable of performing an electron transfer reaction (Faraday reaction) with the electrolyte in the droplet 110 and the electrolyte solution 103, and examples thereof include silver halide and alkali silver halide. Particularly, silver or silver/silver chloride can be used from the viewpoint of potential stability and reliability.

The materials of the individual electrode 112 and the common electrode 105 may be materials serving as a polarization electrode, and for example, gold or platinum can be used. In this case, in order to secure a stable ion current, for example, a substance capable of assisting the electron transfer reaction, such as potassium ferricyanide or potassium ferrocyanide, can be added to the measurement solution. Alternatively, for example, a substance capable of performing an electron transfer reaction such as ferrocenes can be immobilized on a surface of the polarization electrode.

All of the individual electrodes 112 and the common electrode 105 may be made of the above material, or a surface of a base material (copper, aluminum, or the like) may be covered with the above material. Shapes of the individual electrode 112 and the common electrode 105 are not particularly limited, and can be shapes in which a surface area coming into contact with the measurement solution is increased. The individual electrodes 112 and the common electrode 105 are bonded to the wirings, and an electrical signal is sent to a measurement circuit.

The water-repellent liquid 111 is a liquid that has insulating properties and phase-separates from water, and can have high affinity with the biopolymers in some cases. Examples of the water-repellent liquid 111 include silicone oil, fluorine-based oil, mineral oil, and the like. Such liquids are often used in techniques such as PCR and digital PCR. Since the water-repellent liquid 111 is used to convey the droplets 110 by EWOD, a liquid having low viscosity and high fluidity can be used as the water-repellent liquid 111.

Although not illustrated, each of the first liquid tank 104A and the second liquid tank 104B has an injection port for injecting a liquid into the inside and a discharge port for discharging a liquid in the inside.

<Method for Forming Nanopores>

FIG. 3B is a schematic diagram illustrating the biopolymer analysis device 300 in a state in which the nanopores 101 are formed in the thin film 102. In the configuration of FIG. 3A, since the nanopores 101 are not provided, the biopolymers cannot be analyzed. Thus, the nanopores 101 can be formed by applying a voltage value equal to or higher than a dielectric breakdown voltage of the thin film 102 between the plurality of individual electrodes 112 and the common electrode 105.

The method for forming the nanopores 101 on the thin film 102 is not particularly limited, and for example, electron beam irradiation by a transmission electron microscope or the like, dielectric breakdown by voltage application, or the like can be used. For example, the method described in “Itaru Yanagi et al., Sci. Rep. 4, 5000 (2014)” can be used as the method for forming the nanopores 101.

The formation of the nanopores 101 by the voltage application when the thin film 102 is made of Si₃N₄ can be performed in the following procedure, for example. First, the thin film 102 made of Si₃N₄ is hydrophilized by Ar/O₂ plasma (manufactured by Samco Inc.) under the conditions of 10 WW, 20 sccm, 20 Pa, and 45 sec. Subsequently, the biopolymer analysis device 300 including the thin film 102 is set in a flow cell. Thereafter, the individual electrodes 112 and the common electrode 105 are introduced into each of the first liquid tank 104A and the second liquid tank 104B. Then, the droplet 110, which is an electrolyte solution of pH 7.5 containing 1 M of CaCl₂) and 1 mM of Tris-10 mM of EDTA, is conveyed to the first liquid tank 104A, and the second liquid tank 104B is filled with the electrolyte solution.

The voltage is applied not only when the nanopores 101 are formed, but also when the ion current flowing through the nanopores 101 after the nanopores 101 are formed is measured. Here, the first liquid tank 104A positioned on a GND electrode side is referred to as a cis tank, and the second liquid tank 104B positioned on a variable voltage side is referred to as a trans tank. A voltage V_{cis} applied to an electrode on the cis tank side is set to 0 V, and a voltage Vₜᵣₐₙₛ is applied to an electrode on the trans tank side. The voltage Vₜᵣₐₙₛ is generated by, for example, a pulse generator (41501B SMU AND Pulse Generator Expander, manufactured by Agilent Technologies, Inc.).

A current value after pulse application can be read by an ammeter 106 (4156B PRECISION SEMICONDUCTOR ANALYZER, manufactured by Agilent Technologies, Inc.). A process of applying a voltage in order to form the nanopores 101 and a process of reading the ion current value are controlled by, for example, a self-written program (Excel VBA, Visual Basic for Applications) stored in the storage of the computer 108. A current value condition (threshold current) is selected according to a diameter of the nanopore 101 formed before application of a pulse voltage, and a target diameter is obtained while the diameter of the nanopore 101 is sequentially increased.

The diameter of the nanopore 101 can be estimated from the ion current value. A criterion for the condition selection is as represented in Table 1, for example, when the material of the thin film 102 is Si₃N₄ and the thickness of the thin film 102 is 5 nm. Here, an n-th pulse voltage application time tₙ (where, n>2.) is decided by the following Equation.

tₙ=10^{-3+(1/6)(n-1)}−10^{-3+(1/6)(n-2)} For n>2 [Equation 1]

TABLE 1
Voltage application condition

| | | |
|---|---|---|
| Nanopore diameter | Non-opening | φ1.4 nm |
| before pulse voltage | to φ0.7 nm | |
| application | | |
| Applied voltage | 5 | 3.5 |
| (Vₜᵣₐₙₛ) [V] | | |
| Initial application | 0.001 | 0.01 |
| time [s] | | |
| Threshold current | 0.1 nA/0.4 V | 0.4 nA/0.1 V |

The nanopores 101 can be formed not only by the method for applying the pulse voltage but also by electron beam irradiation by TEM (A. J. Storm et al., Nat. Mat. 2 (2003)).

A dimension of the nanopore 101 can be selected according to a type of the biopolymer to be analyzed. The dimension thereof can be, for example, 0.9 nm to 100 nm, and can be 0.9 nm to 50 nm in some cases. Specifically, the dimension of the nanopore 101 is equal to or more than 0.9 nm and equal to or less than 10 nm. For example, the diameter of the nanopore 101 used for analyzing single-stranded DNA having a diameter of about 1.4 nm can be, for example, 0.8 nm to 10 nm or 0.8 nm to 1.6 nm. For example, the diameter of the nanopore 101 used for analyzing double-stranded DNA having a diameter of about 2.6 nm can be 3 nm to 10 nm or 3 nm to 5 nm.

A depth of the nanopore 101 can be adjusted by adjusting the thickness of the thin film 102. The depth of the nanopore 101 can be two times or more a monomer unit constituting the biopolymer, and can be three times or more or five times or more in some cases. For example, when the biopolymer is a nucleic acid, the depth of the nanopore 101 is three or more bases, for example, about 1 nm or more. In this manner, the biopolymers can enter the nanopores 101 while a shape and a moving speed thereof are controlled, and highly sensitive and highly accurate analysis can be performed. The shape of the nanopore 101 is basically circular, and may be elliptical or polygonal.

Immediately before a user analyzes the biopolymers by using the biopolymer analysis device 300, in a state in which the droplets 110 are conveyed to the positions coming into contact with the individual electrodes 112 and are insulated from each other by the water-repellent liquid 111 as illustrated in FIG. 3B, the nanopores 101 are provided in the thin film 102 by the electrical operation, and thus, it is possible to constantly provide the nanopores 101 with high quality.

The biopolymer analysis device 300 may be provided to the user in a state in which the droplets 110 and the water-repellent liquid 111 are conveyed to the positions illustrated in FIG. 3A. Alternatively, the biopolymer analysis device may be provided to the user in a state in which only the water-repellent liquid 111 is introduced into the first liquid tank 104A, and the droplets 110 may be conveyed to the positions illustrated in FIG. 3A by applying the EWOD conveying voltage to the individual electrodes 112 by an operation of the user. The biopolymer analysis device 300 may be provided to the user in a state in which both the first liquid tank 104A and the second liquid tank 104B are empty. In this case, the biopolymer analysis device is in the state illustrated in FIG. 3A by filling the first liquid tank 104A with the water-repellent liquid 111 by an operation of the user, conveying the droplets 110 by the application of the EWOD conveying voltage to the individual electrodes 112, and introducing the electrolyte solution 103 into the second liquid tank 104B.

<Another Configuration Example of Biopolymer Analysis Device>

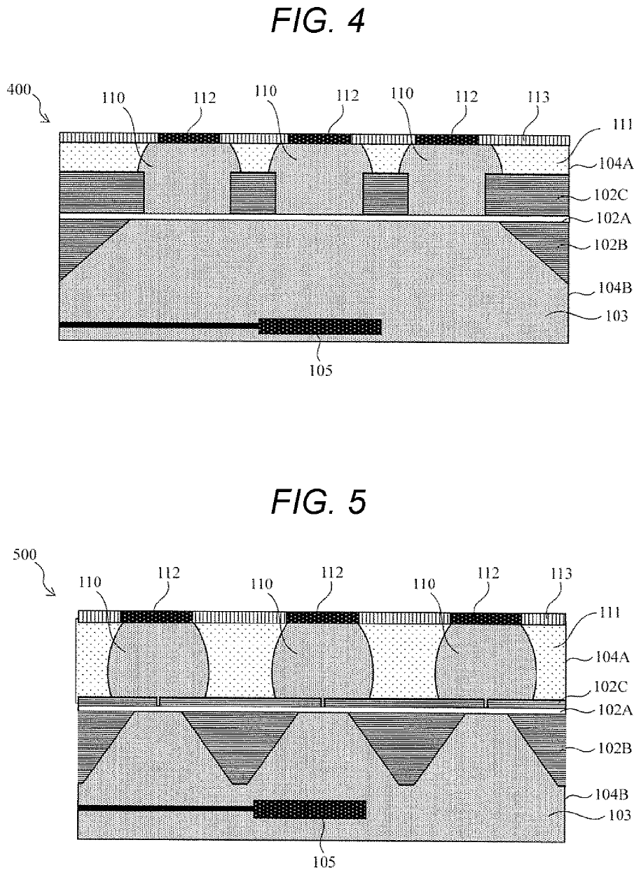

FIG. 4 is a schematic diagram illustrating another biopolymer analysis device 400 according to the first embodiment. The biopolymer analysis device 400 has a configuration adopting the configuration of the present embodiment (FIG. 3) for a typical solid-state nanopore device used for analyzing the biopolymers by a blockade current method. As illustrated in FIG. 4, the biopolymer analysis device 400 includes a thin film 102A made of an inorganic material, a tapered layer 102B disposed on one side of the thin film 102A, and a sacrificial layer 102C disposed on the other side of the thin film 102A. The thin film 102A, the tapered layer 102B, and the sacrificial layer 102C may be collectively referred to as a “thin film”.

Silicon (Si) is generally adopted as the materials of the tapered layer 102B and the sacrificial layer 102C in consideration of mass productivity. The tapered layer 102B is formed by, for example, anisotropic etching of a silicon wafer. The sacrificial layer 102C has a plurality of (three in FIG. 4) etching holes (protrusions) formed by, for example, etching of a silicon wafer at positions facing the plurality of individual electrodes 112, and thus, the thin film 102A is exposed at a plurality of portions to achieve an array. The sacrificial layer 102C supports the thin film 102A by stress. The configuration of such a solid-state nanopore device is described, for example, in U.S. Pat. No. 5,795,782, “Yanagi, et al., Scientific Reports 4, 5000, 2014”, “Akahori, et al., Nanotechnology 25 (27): 275501, 2014”, and “Yanagi, et al., Scientific Reports, 5, 14656, 2015”.

A dimension of the thin film 102A exposed to the droplets 110 needs to be an area in which it is difficult to form two or more nanopores 101 when the nanopores 101 are formed by the application of the voltage, and an area allowable in terms of strength. The area is, for example, about 100 to 500 nm, and a film thickness at which the nanopores 101 having an effective film thickness equivalent to a single base can be formed is appropriately about 3 to 7 nm in order to achieve DNA single base resolution.

As illustrated in FIG. 4, in the case of a configuration in which a plurality of individual solution tanks is arrayed, the exposed portions of the thin film 102 where the nanopores 101 are formed can be regularly sequenced. An interval between the plurality of exposed portions of the thin film 102A can be set to, for example, 0.1 mm to 10 mm or 0.5 mm to 4 mm according to the capability of an electrode and an electric measurement system to be used.

FIG. 5 is a schematic diagram illustrating another biopolymer analysis device 500 according to the first embodiment. As illustrated in FIG. 5, the biopolymer analysis device 500 is different from the biopolymer analysis device 400 illustrated in FIG. 4 in that a plurality of tapered layers 102B is provided. Such a configuration is described in, for example, “Yanagi, et al., Lab on a Chip, 16, 3340-3350, 2016.”.

<Biopolymer Analysis Method>

Hereinafter, a method for continuously performing the formation of the nanopores and the analysis of the biopolymers by using the biopolymer analysis device before the formation of the nanopores will be described. In a biopolymer analysis method according to the present embodiment, any one of the biopolymer analysis devices 300 to 500 illustrated in FIGS. 3A, 4, and 5 may be used, and the common electrode 105 and the plurality of individual electrodes 112 are connected to the ammeter 106, the power supply 107, and the computer 108 illustrated in FIGS. 1 and 2. The biopolymer analysis device in which the first liquid tank 104A and the second liquid tank 104B are empty is used.

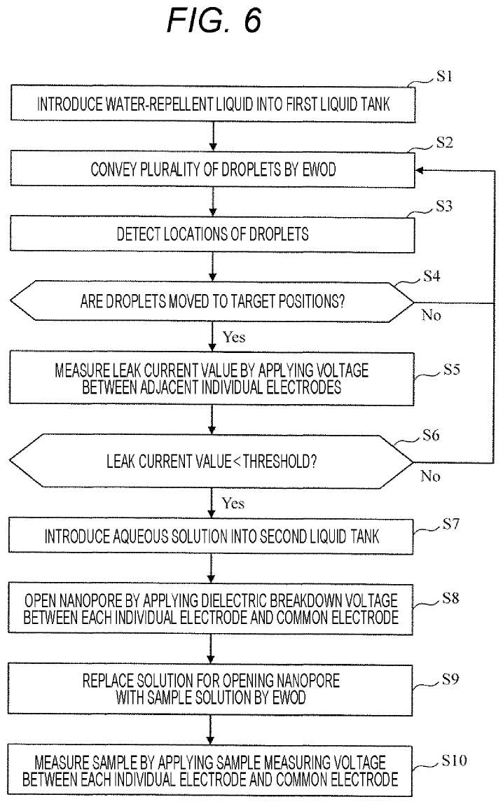

FIG. 6 is a flowchart illustrating the biopolymer analysis method using the biopolymer analysis device according to the present embodiment. First, in step S1, the user introduces the water-repellent liquid 111 from the injection port (not illustrated) of the first liquid tank 104A (individual electrode 112 side), and fills the first liquid tank 104A with the water-repellent liquid 111.

In step S2, the user inputs an operation start instruction to the computer 108, and sequentially injects the plurality of droplets 110 into the injection port (not illustrated) of the first liquid tank 104A. Here, the plurality of droplets 110 is electrolyte solutions for opening the nanopores.

When the operation start instruction is received, the computer 108 applies the EWOD conveying voltage to the individual electrode 112 by the power supply 107, and conveys the plurality of droplets 110 such that each droplet 110 is disposed at the position in contact with one individual electrode 112. The water-repellent liquid 111 prevents the droplets 110 from coming into contact with each other and electrically insulates the droplets 110 from each other. Accordingly, the plurality of independent individual solution tanks (the plurality of channels) each having one individual electrode 112 and one droplet 110 is formed.

In step S3, the computer 108 detects the positions where the plurality of droplets 110 is conveyed. Subsequently, in step S4, the computer 108 determines whether or not the droplets 110 are moved to desired positions. A method for determining the positions of the droplets 110 will be described later. When the droplet 110 does not reach the desired positions (No), the processing returns to step S2, and the computer 108 repeats the conveyance of the droplets 110 until the droplets reach the desired position.

After the droplets 110 reach the desired positions (Yes in step S4), in step S5, the computer 108 applies a voltage for reading a leakage current between the individual electrodes 112 of the adjacent channels, and measures a leakage current value.

In step S6, the computer 108 determines whether or not the measured leakage current value is less than a preset threshold.

When the leakage current value is equal to or more than the threshold (No in step S6), since the channel does not maintain electrical independence, the processing returns to step S2, and the computer 108 tries again from the conveyance of the droplets 110 to the measurement of the leakage current until the leakage current value becomes less than the threshold. Alternatively, instead of returning to step S2, the computer 108 determines that the channel is defective and abandons the use of the channel. At this time, the computer 108 stores the position of the channel determined to be defective in the storage.

When the leakage current value is less than the threshold (Yes in step S6), it can be determined that the channel is favorable, and thus, the processing proceeds to step S7.

After the droplets 110 are moved to all the channels and the electrical independence is confirmed, in step S7, the user introduces the electrolyte solution 103 into the second liquid tank 104B.

In step S8, the computer 108 electrically opens the nanopores 101 by applying a voltage equal to or more than the dielectric breakdown voltage of the thin film 102 between each individual electrode 112 and the common electrode 105. The computer 108 measures current-voltage characteristics of the nanopores 101 by applying a voltage for determining nanopore characteristics between each of the independent individual electrodes 112 and the common electrode 105. Here, when the measured current value falls within a desired current value range, that is, within a desired nanopore diameter range, it is determined that the favorable nanopores 101 are obtained.

When the measured current value is out of the desired range, the computer 108 determines that the channel is a defective portion, and abandons the use of the channel. In this case, the computer 108 stores positional information of the abandoned channel in the storage so as not to move the droplet containing a sample to the abandoned channel. Accordingly, it is possible to prevent a loss of the sample.

Since the droplet 110 conveyed to the individual electrode side by the above operation is the electrolyte solution for opening the nanopores, it is necessary to replace the electrolyte solution with a solution for measuring the sample. In step S9, the computer 108 applies the EWOD conveying voltage to the individual electrode 112, conveys the droplets 110, which are nanopore opening solutions, to the discharge port of the first liquid tank 104A, and moves the droplets to a waste liquid tank (not illustrated) connected to the discharge port.

Thereafter, the user injects the droplets (sample solutions) for measuring the sample containing the biopolymers from the injection port of the first liquid tank 104A, and the computer 108 moves the sample solution to a portion where the favorable nanopores 101 are formed by applying the EWOD conveying voltage to the individual electrode 112.

After all the sample solutions are conveyed, in step S10, the computer 108 measures the sample by applying the sample measuring voltage between each individual electrode 112 and the common electrode 105.

When the sample is replaced, an operation similar to that in step S9 is executed. Specifically, the computer 108 applies the EWOD conveying voltage to the individual electrode 112, conveys the sample solution for which measurement is completed to the discharge port of the first liquid tank 104A, and moves the sample solution to the waste liquid tank connected to the discharge port. Thereafter, the user introduces a new sample solution from the injection port of the first liquid tank 104A, and the computer 108 conveys the new sample solution by applying the EWOD conveying voltage to the individual electrode 112. As described above, the solution in each individual solution tank can be smoothly replaced by the EWOD.

<Method for Determining Position of Droplet>

Next, a method for detecting the positions of the droplets 110 in steps S3 and S4 described above will be described. Whether or not the droplets 110 reach the desired positions can be detected by various methods. For example, a transparent substrate and transparent electrodes are used as the substrate 113 and the individual electrodes 112, and an observation device such as a microscope (a mechanism for determining whether or not the plurality of droplets is conveyed to the desired positions) is provided above the individual electrodes 112 and the substrate 113. In this manner, it is possible to optically observe the images of inside of the first liquid tank 104A. The observation device is configured to be able to transmit image data obtained by imaging an observation portion to the computer 108. The computer 108 can determine the positions of the droplets 110 based on the image data.

On the other hand, when opaque materials are used for the individual electrodes 112 and the substrates 113, the images of the droplets 110 cannot be observed. In this case, the positions of the droplets 110 can be determined by using an electrical method instead of the optical method described above. Since the droplets 110 conveyed by the biopolymer analysis device according to the present embodiment contain the electrolyte, the droplets are electrically conducted. Thus, it is possible to determine whether or not the droplets 110 come into contact with the individual electrodes 112 (the droplets 110 are at the positions of the individual electrodes 112) by applying an electrical operation between the individual electrodes 112 or between each individual electrode 112 and the common electrode 105 and examining whether or not an electrical reaction changes.

For example, impedance characteristics at the time of AC application vary depending on whether the individual electrodes 112 come into contact with the water-repellent liquid 111 containing the electrolyte or the electrolyte solution. Accordingly, it can be determined whether or not the droplets 110 come into contact with the individual electrode 112 by applying the alternating current to the individual electrodes 112 and measuring the impedance.

Alternatively, the positions of the droplets 110 can also be determined by measuring the current value between the individual electrode 112 and the common electrode 105 and examining resistance characteristics. For example, when the water-repellent liquid 111 comes into contact with the individual electrodes 112 and the thin film 102, since the individual electrodes 112 and the common electrode 105 are completely insulated from each other by high insulating properties of the water-repellent liquid 111, the observed current value is 10⁻¹³ to 10⁻¹⁴ Å or less. On the other hand, in a state in which the electrolyte solutions such as the droplets 110 come into contact with the individual electrodes 112 and the thin film 102, since the electrolyte solution is a low resistor, a current value of 10⁻¹¹ to 10⁻¹² Å is observed between the individual electrodes 112 and the common electrode 105 even before the nanopores 101 are opened. A case where such a current value is observed is reported in, for example, “Scientific Reports, 5, 14656, 2015, Yanagi, et al.”. As described above, it is possible to determine whether the droplets 110 come into contact with the individual electrodes 112 and the thin film 102 by detecting a difference between the current values, and thus, it is possible to determine the positions of the droplets 110.

### Technical Effects

As described above, in the first embodiment, the plurality of droplets 110 is automatically moved to the desired positions by applying the EWOD conveying voltage to the individual electrode 112, and thus, it is possible to collectively inject the solutions into the plurality of independent individual solution tanks. At this time, the droplets 110 are electrically insulated from each other by the presence of the water-repellent liquid 111, and the electrical independence is maintained. When the solution is replaced, since the droplets 110 are conveyed by the EWOD and abandoned and new droplets 110 are similarly conveyed to desired positions, the solution can be smoothly replaced. Accordingly, it is possible to achieve both the collective injection of the solutions into the plurality of independent individual solution tanks and the solution replacement of the individual solution tanks while maintaining the insulation between the parallel channels. Since a liquid feeding device for conveying or replacing the solution is unnecessary, an increase in size of the device and an increase in installation cost can be avoided.

The EWOD exhibits an effect even when a degree of integration is high, that is, when a component dimension is minute. In particular, since the EWOD can convey even microdroplets of several μL to several nL, it is possible to measure the sample with the small amount of droplets.

In the biopolymer analysis device according to the present embodiment, the independent individual solution tanks can be integrated. Accordingly, it is possible to simultaneously measure different types of samples. For example, a certain droplet as a solution of a sample A and another droplet as a solution of a sample B are prepared, and the droplets are conveyed to appropriate positions. Thus, samples of different types can be simultaneously measured. When the biopolymer analysis device according to the present embodiment is used as, for example, a DNA sequencer, the sample A having a genetic mutation A and the sample B having a genetic mutation B can be separately and simultaneously measured on one device. The same applies to a gene detection method based on hybridization with a probe fixed. Alternatively, DNA sequencing and the above-described hybridization detection method or the like can be performed simultaneously. As described above, the throughput of the measurement can be improved by integrating the individual solution tanks.

### Second Embodiment

In general, when the droplets are conveyed by the EWOD, an insulator (dielectric) may be installed on the electrode surface in order to enhance wettability to the electrode surface by drawing and polarizing electric charges from the surface of the droplet. However, when the insulator is installed on the surface of the individual electrode 112 of the first embodiment, it is difficult to measure the current due to high insulation resistance, and the biopolymers cannot be analyzed by using the individual electrode 112.

In order to solve such a problem, in a second embodiment, as individual electrodes, two types of electrodes of one or more electrodes for current measurement and electrodes for EWOD are separately installed for the droplets.

<Configuration Example of Biopolymer Analysis Device>

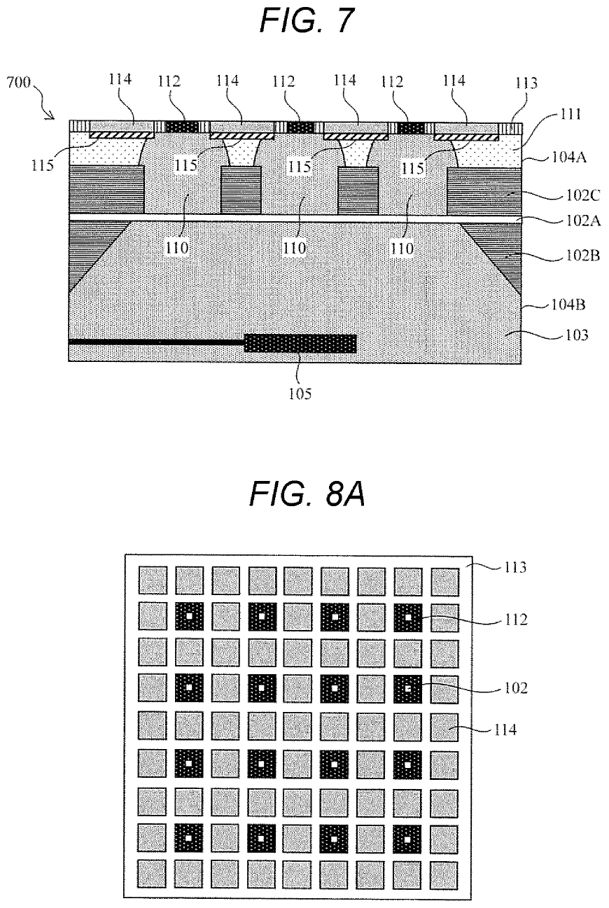

FIG. 7 is a schematic diagram illustrating a biopolymer analysis device 700 according to the second embodiment. The biopolymer analysis device 700 is different from the biopolymer analysis device 400 illustrated in FIG. 4 in the configuration of a substrate 113. Accordingly, configurations other than the substrate 113 are not described.

As illustrated in FIG. 7, a plurality of individual electrodes 112 (a plurality of third electrodes) for current measurement and a plurality of EWOD electrodes 114 (a plurality of first electrodes) are embedded in the substrate 113. The plurality of individual electrodes 112 is arranged at positions facing exposed portions of the thin film 102A. Insulators 115 are provided on inner surfaces of the EWOD electrodes 114. As will be described later, the plurality of EWOD electrodes 114 is arranged so as to form lanes for conveying droplets 110 at positions coming into contact with the individual electrodes 112.

FIG. 7 illustrates a state in which the droplets 110 are conveyed to desired positions, and each droplet 110 comes into contact with at least one individual electrode 112 and the plurality of EWOD electrodes 114 surrounding the individual electrode. In this manner, EWOD conveyance and current measurement can be performed without problems by providing the electrodes for current measurement and the EWOD electrodes as separate applications.

<Biopolymer Analysis Method>

The biopolymer analysis method according to the present embodiment is substantially the same as that of the first embodiment (FIG. 6), but is different from that of the first embodiment in that an EWOD conveying voltage is applied to the EWOD electrodes 114 instead of the individual electrodes 112 in the conveyance of the droplets in steps S2 and S9.

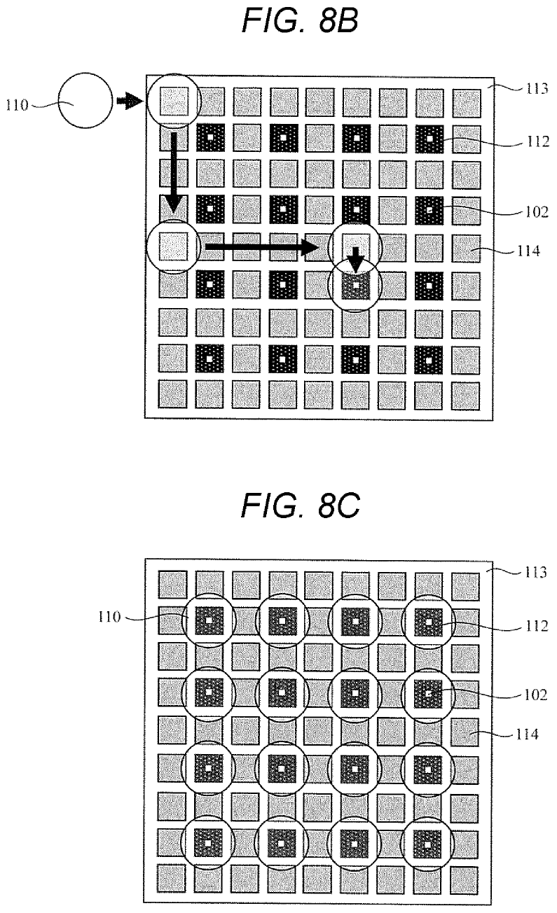

FIG. 8A is a top view of the biopolymer analysis device 700. As illustrated in FIG. 8A, on the substrate 113, a total of 16 individual electrodes 112 for current measurement of 4 columns×4 rows are arranged, and the plurality of EWOD electrodes 114 is arranged around each individual electrode 112. Thus, the plurality of EWOD electrodes 114 form a lane for conveying the droplet 110, and can smoothly convey the droplet 110. Each individual electrode 112 is disposed above the exposed portion of the thin film 102. When each individual electrode 112 is a transparent electrode, as illustrated in FIG. 8A, the thin film 102 can be observed from above the individual electrode 112. The state illustrated in FIG. 8A is a state after the water-repellent liquid 111 is introduced in step S1 (FIG. 6) described in the first embodiment.

FIGS. 8B and 8C are top views of the biopolymer analysis device 700 illustrating a scene in which the droplet 110 is conveyed. As described above, the droplet 110 is conveyed by applying the EWOD conveying voltage to the EWOD electrodes 114. As illustrated in FIG. 8B, for example, when the droplets 110 conveyed via a channel of a flow cell are introduced into the first liquid tank 104A and come into contact with the EWOD electrodes 114 to which the EWOD conveying voltage is applied, the droplets 110 can be transported discretely by one electrode. Finally, one droplet 110 is disposed between the thin film 102 and the individual electrode 112. As illustrated in FIG. 8C, the droplets 110 can be arranged between all the exposed portions of the thin film 102 and the individual electrodes 112 by similarly repeating this operation.

The numbers and arrangement layouts of the individual electrodes 112 and the EWOD electrodes 114 are not limited to those illustrated in FIGS. 8A to 8C, and can be appropriately changed. For example, when the channels are highly integrated, the individual electrodes 112 may be provided in units of several hundreds to several thousands or more.

### Technical Effects

As described above, in the present embodiment, the configuration in which the individual electrode 112 for current measurement and the EWOD electrodes 114 are provided in the first liquid tank 104A is adopted. Accordingly, even though the insulators 115 are provided on surfaces of the EWOD electrodes 114, the formation of the nanopores and the measurement of the current can be performed without any problem by using the individual electrodes 112.

### Third Embodiment

As described above, when the insulators (dielectrics) are installed on the surfaces of the individual electrodes 112 of the first embodiment, it is difficult to measure the current due to the high insulation resistance, and the biopolymers cannot be analyzed by using the individual electrodes 112.

In order to solve such a problem, in a third embodiment, a circuit for EWOD conveyance, a circuit for nanopore opening, and a circuit for current measurement are connected to each individual electrode 112, and a voltage applied to the individual electrode 112 is controlled by switching between these circuits.

<Configuration Example of Biopolymer Analysis Device>

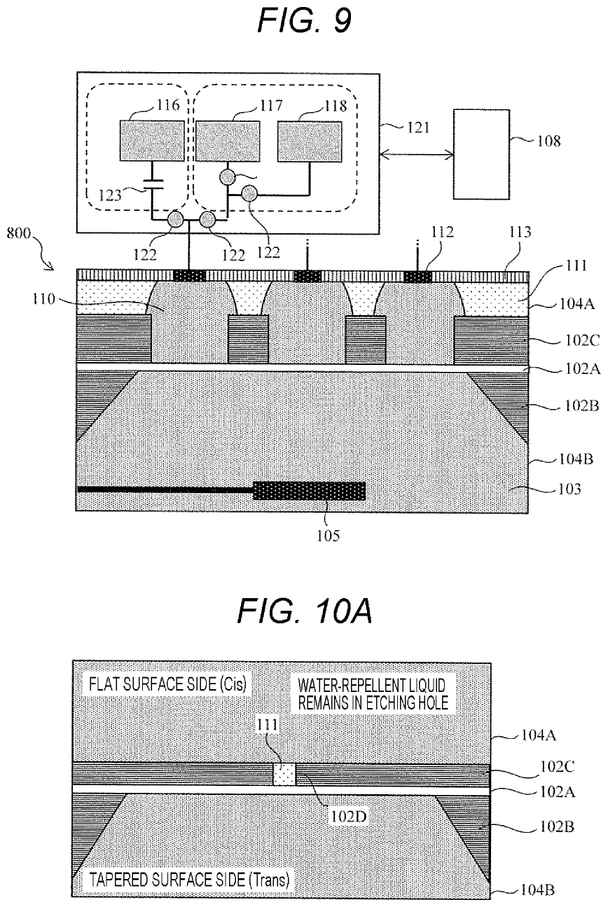

FIG. 9 is a schematic diagram illustrating a biopolymer analysis device 800 according to the third embodiment. A configuration of the biopolymer analysis device 800 is substantially the same as that of the biopolymer analysis device 400 of FIG. 4 described in the first embodiment, but a control circuit 121 (controller) is connected to the individual electrodes 112 (the plurality of first electrodes) through wirings. As illustrated in FIG. 9, in the control circuit 121, an EWOD conveying circuit 116, a nanopore opening circuit 117, a current measuring circuit 118, and a plurality of switches 122 for switching between these circuits are provided. The control circuit 121 is connected to the computer 108 (controller). The switching of the switches 122 and the application of the voltage using the circuits 116 to 118 are controlled by the computer 108.

For example, a circuit having a configuration such as a capacitor 123 (insulator) that appropriately draws electric charges from the droplets between the EWOD conveying circuit 116 and the individual electrodes 112 is provided, and thus, the EWOD conveyance can be appropriately performed without installing the insulators on the surfaces of the individual electrodes 112. One EWOD conveying circuit 116 common to all the individual electrodes 112 may be provided.

<Biopolymer Analysis Method>

The biopolymer analysis method according to the present embodiment is substantially the same as that of the first embodiment (FIG. 6), but is different from that of the first embodiment in that the computer 108 changes the voltage applied to the individual electrode 112 by switching between the switches 122. Accordingly, only differences from the first embodiment will be described.

In step S2, the computer 108 connects the EWOD conveying circuit 116 and each individual electrode 112 by switching between the switches 122, and applies the EWOD conveying voltage to each individual electrode 112.

In step S5, the computer 108 connects the current measuring circuit 118 and each individual electrode 112 by switching between the switch 122, applies a voltage for reading the leakage current between the individual electrodes 112 of the adjacent channels, and measures a leakage current value.

In step S8, the computer 108 connects the nanopore opening circuit 117 and each individual electrode 112 by switching between the switches 122, applies a voltage equal to or more than the dielectric breakdown voltage of the thin film 102 between each individual electrode 112 and the common electrode 105, and electrically opens the nanopores 101.

In step S9, the computer 108 connects the EWOD conveying circuit 116 and each individual electrode 112 by switching between the switches 122. Subsequently, the EWOD conveying voltage is applied to the individual electrode 112, the droplets 110, which are the nanopore opening solutions, are conveyed to the discharge port of the first liquid tank 104A, and the droplets are moved to a waste liquid tank (not illustrated) connected to the discharge port.

In step S10, the computer 108 connects the current measuring circuit 118 and each individual electrode 112 by switching between the switches 122, applies the sample measuring voltage between each individual electrode 112 and the common electrode 105, and measures the sample.

### Technical Effects

As described above, in the present embodiment, the configuration in which the EWOD conveying circuit 116, the nanopore opening circuit 117, and the current measuring circuit 118 are connected to the plurality of individual electrodes 112, and the circuits connected to the individual electrodes 112 are switched by the switches 122 is adopted. Accordingly, it is possible to convey the droplets 110, form the nanopores, and measure the current value only with the individual electrodes 112 and the common electrode 105 without separately providing the EWOD electrode. Therefore, it is possible to increase the number of channels per unit area of the biopolymer analysis device as compared with the second embodiment.

### Fourth Embodiment

As illustrated in FIGS. 4 and 5, the solid-state nanopore device often has a structure in which the sacrificial layer 102C that is a flat surface is provided on one side of the thin film 102A and the tapered layer 102B that is a tapered surface is provided on the other side. However, the sacrificial layer 102C has a structure (etching hole) in which only a specific region is removed by chemical etching or dry etching in order to expose the thin film 102A.

In some structures of the biopolymer analysis device, the water-repellent liquid 111 remains in the etching hole, and the droplet 110 cannot enter the etching hole. Thus, a problem of a defective channel is caused.

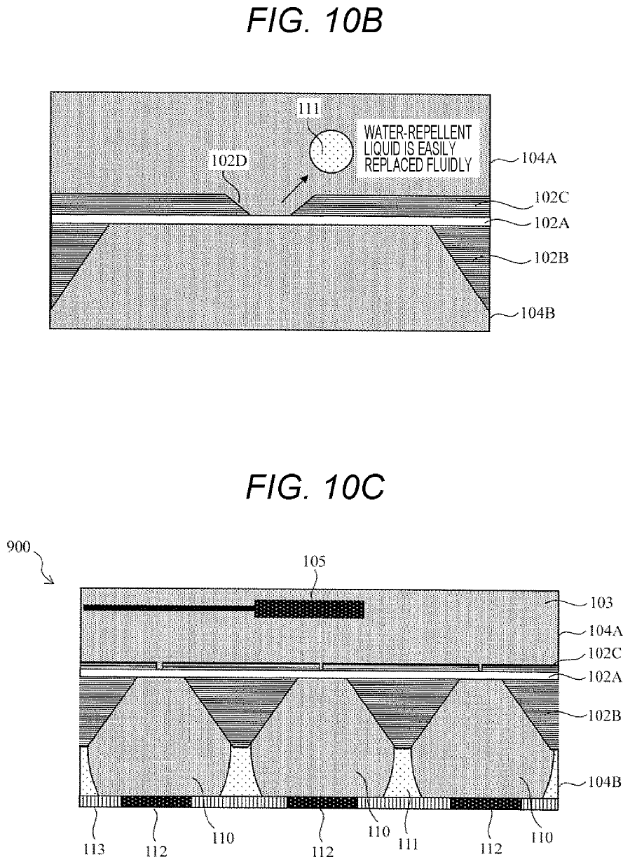

FIG. 10A is a schematic diagram illustrating a state in which the water-repellent liquid 111 remains in an etching hole 102D of the sacrificial layer 102C. As illustrated in FIG. 10A, when the etching hole 102D has, for example, a cylindrical shape, the water-repellent liquid 111 enters first, and this space is a hydrodynamically immovable region. Thus, when the droplet 110 is conveyed onto the etching hole 102D, the replacement is not promptly preformed fluidly, and the water-repellent liquid 111 remains in the etching hole 102D. Such a phenomenon easily occurs in the water-repellent liquid often used in the EWOD. That is, since the water-repellent liquid has chemical properties such as low viscosity and low surface tension, a phenomenon in which the replacement is not performed in the structure having the immovable region like the cylindrical etching hole 102D occurs. In particular, when the density of the water-repellent liquid 111 is higher than the density of the droplet, buoyancy acts reversely to the replacement, and thus, the replacement becomes more difficult.

A configuration for preventing the water-repellent liquid 111 from remaining in the etching hole 102D of the sacrificial layer 102C will be described below.

FIG. 10B is a schematic diagram illustrating a structure of the sacrificial layer 102C of the present embodiment. As illustrated in FIG. 10B, in the sacrificial layer 102C of the present embodiment, a cross-sectional shape of the etching hole 102D (recess) is formed in a tapered shape. In this manner, the cross-sectional shape of the etching hole 102D is formed so as not to have the fluidly immovable region such as the tapered shape, and thus, the water-repellent liquid 111 can be easily replaced fluidly by the droplet as the electrolyte solution.

When the etching hole 102D has the cylindrical shape, the electrolyte solution is filled in the cylindrical etching hole 102D in advance before the first liquid tank 104A is filled with the water-repellent liquid 111, and thus, the water-repellent liquid 111 can be prevented from remaining. Since the liquid in the cylindrical etching hole 102D is less likely to be replaced fluidly, the water-repellent liquid 111 does not enter the etching hole 102D when the water-repellent liquid 111 subsequently moves. In this case, the water-repellent liquid 111 is less likely to enter the etching hole 102D by using a fluid having a specific gravity lower than that of water as the water-repellent liquid 111.

FIG. 10C is a schematic diagram illustrating another biopolymer analysis device 900 according to the present embodiment. As illustrated in FIG. 10C, structures of a thin film 102A, a tapered layer 102B, and a sacrificial layer 102C of the biopolymer analysis device 900 are similar to those of the biopolymer analysis device 500 of the first embodiment (FIG. 5), but a substrate 113 on which the plurality of individual electrodes 112 is provided is disposed in a second liquid tank 104B, and a common electrode 105 is disposed in a first liquid tank 104A. A plurality of droplets 110 and a water-repellent liquid 111 are introduced into the second liquid tank 104B, and an electrolyte solution 103 is introduced into the first liquid tank 104A.

In this manner, the water-repellent liquid 111 can be fluidly and easily replaced with the droplets 110 by filling the tapered layer 102B side (second liquid tank 104B) with the water-repellent liquid 111 and then conveying the droplets 110.

### Technical Effects

As described above, in the present embodiment, the configuration in which the cross-sectional shape of the etching hole 102D formed in the sacrificial layer 102C is the tapered shape is adopted. Alternatively, the configuration in which the cylindrical etching hole 102D is filled with the electrolyte solution in advance is adopted. It is also possible to adopt the configuration in which the plurality of individual electrodes 112 is provided on the tapered layer 102B side (second liquid tank 104B) and the water-repellent liquid 111 and the droplets 110 are introduced into the tapered layer 102B side. In this manner, it is possible to prevent the water-repellent liquid 111 from remaining in the etching hole 102D formed in the sacrificial layer 102C and from being the defective channel.

### Fifth Embodiment

<Configuration Example of Biopolymer Analysis Device>

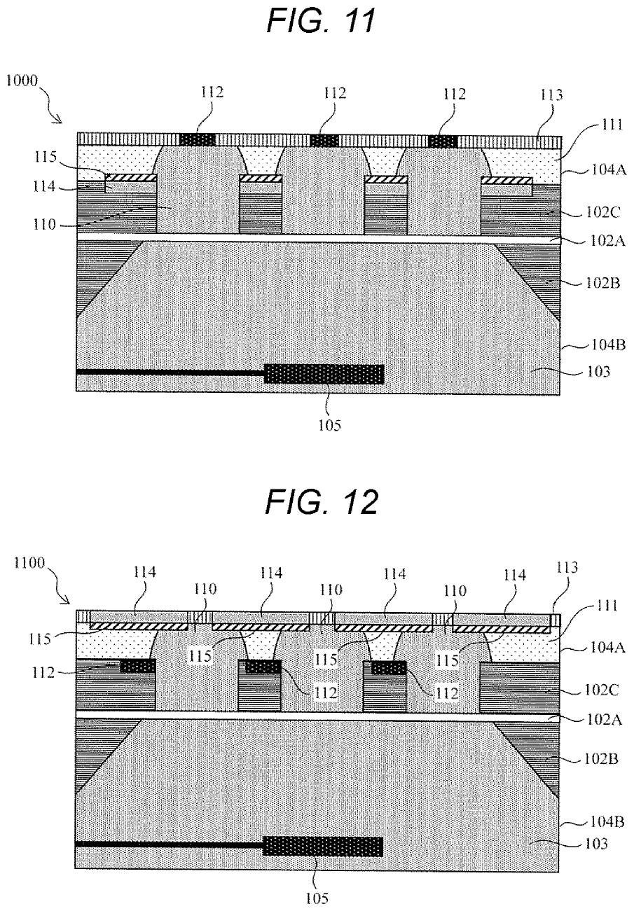

FIG. 11 is a schematic diagram illustrating a biopolymer analysis device 1000 according to a fifth embodiment. As illustrated in FIG. 11, the biopolymer analysis device 1000 according to the present embodiment is different from the first embodiment (FIG. 4) and the second embodiment (FIG. 7) in that EWOD electrodes 114 are formed on an upper surface of a sacrificial layer 102C (thin film). Insulators 115 are arranged on surfaces of the EWOD electrodes 114. Each EWOD electrode 114 is connected to an external circuit through a wiring (not illustrated) provided inside the sacrificial layer 102C. Droplets 110 are conveyed to positions coming into contact with one individual electrode 112 and coming into contact with at least two adjacent EWOD electrodes 114.

FIG. 12 is a schematic diagram illustrating another biopolymer analysis device 1100 according to the fifth embodiment. As illustrated in FIG. 11, the biopolymer analysis device 1100 according to the present embodiment is different from the first embodiment (FIG. 4) and the second embodiment (FIG. 7) in that a plurality of individual electrodes 112 (a plurality of third electrodes) for current measurement is formed on an upper surface of a sacrificial layer 102C (thin film), and only EWOD electrodes 114 (a plurality of first electrodes) are formed on a substrate 113. Each individual electrode 112 is connected to an external circuit through a wiring (not illustrated) provided inside the sacrificial layer 102C. Droplets 110 are conveyed to positions coming into contact with one individual electrode 112 and coming into contact with at least two adjacent EWOD electrodes 114. In other words, each of the individual electrodes 112 is disposed so as to come into contact with one droplet 110.

### Technical Effects

As described above, each of the biopolymer analysis devices 1000 and 1100 according to the present embodiment includes the individual electrodes 112 for current measurement and the EWOD electrodes 114, and adopt the configuration in which any one of the individual electrodes 112 or the EWOD electrodes 114 are integrated with the sacrificial layer 102C on the thin film 102A. Accordingly, as compared with the case where both the individual electrodes 112 for current measurement and the EWOD electrodes 114 are provided on the substrate 113 as in the second embodiment, the channels can be further integrated, and the measurement using the droplets having a smaller volume can be performed.

### Sixth Embodiment

In the first embodiment, as illustrated in FIG. 3A, the configuration in which the substrate 113 having the plurality of individual electrodes 112 is disposed on one side (first liquid tank 104A) of the thin film 102 and the droplets 110 are introduced has been described. On the other hand, in a sixth embodiment, substrates 113 having a plurality of individual electrodes 112 are disposed on both sides (a first liquid tank 104A and a second liquid tank 104B) of a thin film 102, and droplets 110 are introduced into both the first liquid tank 104A and the second liquid tank 104B.

<Configuration Example of Biopolymer Analysis Device>

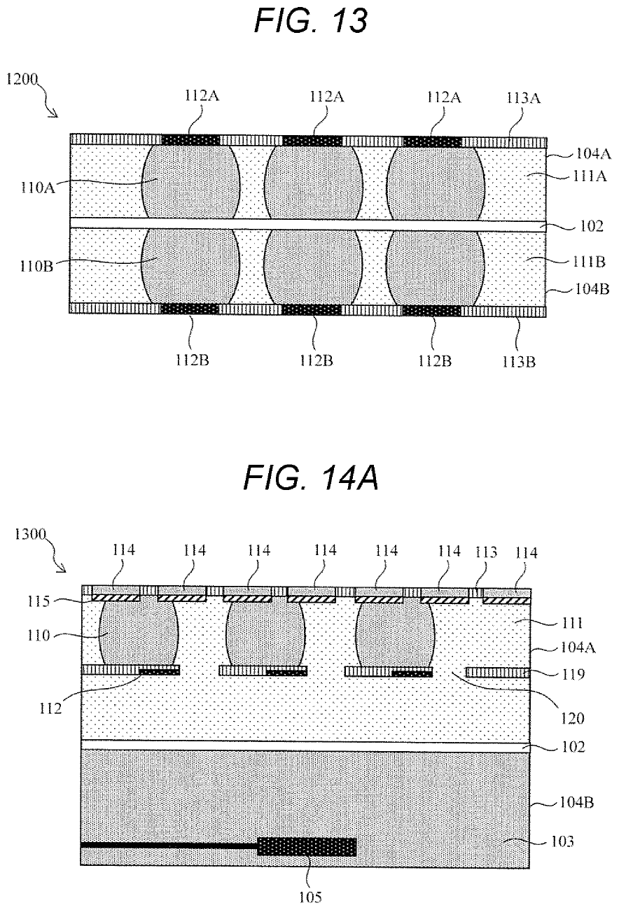

FIG. 13 is a schematic diagram illustrating a biopolymer analysis device 1200 according to the sixth embodiment. As illustrated in FIG. 13, the biopolymer analysis device 1200 according to the present embodiment includes a thin film 102, a first liquid tank 104A, a second liquid tank 104B, a substrate 113A having a plurality of individual electrodes 112A (a plurality of first electrodes), and a substrate 113B having a plurality of individual electrodes 112B (a plurality of second electrodes). The substrate 113A is provided in the first liquid tank 104A, and the substrate 113B is provided in the second liquid tank 104B. The plurality of individual electrodes 112A and the plurality of individual electrodes 112B are arranged at positions facing each other with the thin film 102 interposed therebetween.

A plurality of droplets 110 (measurement solutions) and a water-repellent liquid 111 are introduced into the first liquid tank 104A and the second liquid tank 104B, respectively. Each droplet 110 is electrically insulated from the adjacent droplet 110 by the water-repellent liquid 111, and the droplets are independent of each other. The plurality of droplets 110 comes into contact with the individual electrode 112, respectively, and thus, an electrical operation such as application of a voltage can be performed on each droplet 110. Other configurations are similar as those of the biopolymer analysis device 300 (FIG. 3) according to the first embodiment, and thus, the description thereof is omitted.

<Biopolymer Analysis Method>

Since a biopolymer analysis method according to the present embodiment is substantially the same as that of the first embodiment, the biopolymer analysis method according to the present embodiment will be described with reference to FIG. 6. Steps similar to those in the first embodiment will not be described.

First, steps S1 to S6 of the first embodiment are performed, and the plurality of individual solution tanks is formed by introducing the water-repellent liquid 111 and the droplets 110 into the first liquid tank 104A. Thereafter, instead of step S7, the plurality of individual solution tanks is formed by introducing the water-repellent liquid 111 and the droplets 110 into the second liquid tank 104B as in steps S1 to S6.

Subsequently, in step S8, the computer 108 electrically opens the nanopores 101 by applying the voltage equal to or more than the dielectric breakdown voltage of the thin film 102 between the individual electrodes 112A and the individual electrodes 112B facing each other.

In steps S9 and S10, the droplets 110 for opening the nanopores are abandoned from the first liquid tank 104A by applying the EWOD conveying voltage to the individual electrodes 112A, the sample is measured by introducing the sample solution, and then the droplets 110 for opening the nanopores are replaced with the sample solution by similarly applying the EWOD conveying voltage to the individual electrodes 112B in the second liquid tank 104B. Thereafter, the sample can be measured for the sample solution introduced into the second liquid tank 104B by reversing the voltage applied between the individual electrodes 112A and the individual electrodes 112B facing each other.

### Technical Effects

As described above, in the present embodiment, the configuration in which the substrates 113 having the plurality of individual electrodes 112 are provided in both the first liquid tank 104A and the second liquid tank 104B and the droplets 110 are conveyed by the EWOD is adopted. Accordingly, as compared with the first embodiment in which the sample solution is introduced into only one liquid tank (first liquid tank 104A), the number of samples can be measured twice without performing the replacement of the sample solution.

### Seventh Embodiment

In the first embodiment, the configuration in which the first liquid tank 104A is one layer has been described. The inside of the first liquid tank 104A may have a two-layer structure of a layer for conveying the droplets 110 and a layer for measuring the sample.

<Configuration Example of Biopolymer Analysis Device>

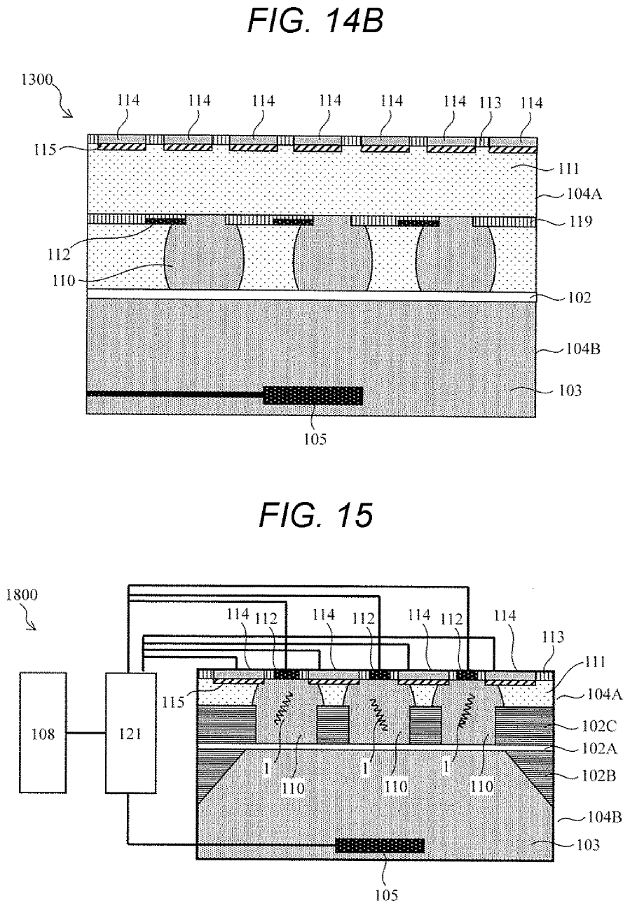

FIG. 14A is a schematic diagram illustrating a biopolymer analysis device 1300 according to a seventh embodiment. As illustrated in FIG. 14A, in the biopolymer analysis device 1300 according to the present embodiment, a substrate 113 forming an upper surface of a first liquid tank 104A is disposed, a substrate 119 is disposed substantially parallel to the substrate 113 inside the first liquid tank 104A, and the first liquid tank 104A has a two-layer structure. A plurality of EWOD electrodes 114 (a plurality of first electrodes) is provided in the substrate 113. Each of the plurality of EWOD electrodes 114 is covered with insulators 115. A plurality of individual electrodes 112 (a plurality of third electrodes) and a plurality of openings 120 through which droplets 110 conveyed between the substrate 113 and the substrate 119 can pass are provided in the substrate 119.

The droplets 110 are conveyed by filling the first liquid tank 104A with a water-repellent liquid 111, introducing a plurality of droplets 110 into an upper layer (between the substrate 113 and the substrate 119) of the first liquid tank 104A, and applying an EWOD conveying voltage between the adjacent EWOD electrodes 114. When the droplets 110 are conveyed to positions of the openings 120, the droplets 110 move to a lower layer (between the substrate 119 and a thin film 102) via the openings 120. The droplets 110 can move from the upper layer to the lower layer of the first liquid tank 104A by using gravity, buoyancy, or a difference in surface tension of a substrate surface with respect to water.

A hydrophilization treatment may be performed on the substrate 119 on a wall surface of the opening 120. Accordingly, this makes it easier to move the droplets 110 to the lower layer.

FIG. 14B is a schematic diagram illustrating a state in which the plurality of droplets 110 is arranged in the lower layer of the first liquid tank 104A. As illustrated in FIG. 14B, each individual electrode 112 is disposed so as to come into contact with one droplet 110 when each droplet 110 passes through the opening 120 and moves to the lower layer. In this manner, the nanopores can be opened in the thin film 102 and the sample can be measured by forming an individual solution tank in which one individual electrode 112 comes into contact with one droplet 110 and applying a dielectric breakdown voltage or a current measuring voltage between the individual electrode 112 and the common electrode 105.

### Technical Effects

As described above, the biopolymer analysis device according to the present embodiment has a two-layer structure in which the substrate 113 having the plurality of EWOD electrodes 114 and the substrate 119 having the plurality of individual electrodes 112 are provided in the first liquid tank 104A. Accordingly, the plurality of EWOD electrodes 114 and the plurality of individual electrodes 112 can be arranged at higher density on each of the substrates 113 and 119 as compared with the second embodiment that adopts the configuration in which both the plurality of EWOD electrodes 114 and the plurality of individual electrodes 112 are provided on the substrate 113.

### Eighth Embodiment

In the first embodiment to the seventh embodiment, the configuration of the biopolymer analysis device has been mainly described. Hereinafter, in the present embodiment, a biopolymer analysis equipment using the biopolymer analysis device will be described. Any one of the biopolymer analysis devices according to the first embodiment to the seventh embodiment may be used as the biopolymer analysis device included in the biopolymer analysis equipment.

<Configuration Example of Biopolymer Analysis Equipment>

FIG. 15 is a schematic diagram illustrating a configuration example of biopolymer analysis equipment 1800. As an example, the biopolymer analysis equipment 1800 includes the biopolymer analysis device 700 according to the second embodiment (see FIG. 7), a control circuit 121, and a computer 108 (controller).

As illustrated in FIG. 15, a plurality of droplets 110 (sample solution) containing biopolymers 1 is conveyed to a first liquid tank 104A. Nanopores are not formed in a thin film 102A. An electrolyte solution 103 is introduced into a second liquid tank 104B. In this manner, the nanopores can be formed in the thin film 102A by using the droplets 110 containing the biopolymers 1, and the biopolymers 1 can be analyzed as it is. In this case, since it is not necessary to replace a solution for opening the nanopores with a sample solution, a measurement time can be shortened.

Although not illustrated, an EWOD conveying circuit, a nanopore opening circuit, a current measuring circuit, and a switch for switching between these circuits are provided inside the control circuit 121. Each individual electrode 112 and a common electrode 105 are connected to the nanopore opening circuit and the current measuring circuit via wirings. EWOD electrodes 114 are connected to the EWOD conveying circuit via wirings.

An ammeter that measures an ion current (blockade current) flowing between each individual electrode 112 and the common electrode 105 is provided in the current measuring circuit. The ammeter includes an amplifier that amplifies the current flowing between the individual electrode 112 and the common electrode 105, and an analog-to-digital converter. The ammeter is connected to the computer 108, and the analog-to-digital converter outputs, as a digital signal, a value of the detected ion current to the computer 108.

The computer 108 is, for example, a terminal such as a personal computer, a smartphone, or a tablet, and includes a data processing unit that processes various kinds of data and a storage that stores an output value of the ammeter, data calculated by the data processing unit, and the like. The data processing unit counts the biopolymers 1 and acquires monomer sequence information of the biopolymers 1 based on a current value of the ion current (blockade current) output from the ammeter. The data processing unit determines whether or not leakage occurs at the positions of the droplets 110 or between the droplets 110 and whether or not the nanopores are formed in the thin film 102 based on electrical characteristics such as a measured current value.

The computer 108 controls switching of the control circuit 121 between switches and application of a voltage to the common electrode 105, each individual electrode 112, and each EWOD electrode 114.

The control circuit 121 and the computer 108 may be integrated with the biopolymer analysis device instead of providing the control circuit 121 and the computer 108 separately from the biopolymer analysis device 700 as illustrated in FIG. 15.

<Analysis of Biopolymer>

In the state illustrated in FIG. 15, when a voltage for opening the nanopores is applied between each individual electrode 112 and the common electrode 105, the nanopores are formed in the thin film 102A. Thereafter, when a current measuring voltage is subsequently applied between the individual electrode 112 and the common electrode 105, a potential difference is generated between both surfaces of the thin film 102A, and the biopolymer 1 dissolved in the droplet 110 is migrated toward the common electrode 105. When the biopolymer 1 is DNA, since the biopolymer is negatively charged in the droplet 110, the biopolymer 1 can be migrated toward the common electrode 105 by using the common electrode 105 as a positive electrode. When the biopolymer 1 passes through the nanopore, a blockade current flows.

In the measurement of the blockade current using the biopolymer analysis device, a current value measured in the absence of the biopolymer 1 is used as a reference (pore current), a decrease in current observed when the nanopore surrounds the biopolymer 1 (blockade of the nanopore by the biopolymer 1) is measured, and a passage speed and state of a molecule are observed. When the biopolymer 1 finishes passing through the nanopore, the acquired current value returns to the pore current. From this blockade time, a nanopore passage speed of the biopolymer 1 can be analyzed, and characteristics of the biopolymer 1 can be analyzed from a blockade amount.

In the nanopore method for analyzing the biopolymer by the electrical signal, particularly a signal change of the ion current, as the electrical conductivity of the electrolyte solution is higher, a signal change amount of the ion current is larger. Thus, it is possible to perform measurement at a high SN ratio. Although depending on the transference number of ionic species and the like, generally, the electrical conductivity of the electrolyte solution can be increased by increasing ionic strength, that is, salt concentration. Accordingly, in the nanopore analysis, measurement is performed at a highest possible salt concentration from the viewpoint of the SN ratio. In particular, in the nanopore analysis, a potassium chloride aqueous solution having a concentration of 1 M is often used, and a high salt concentration condition having an ionic strength of 3 M or more is used in some cases. A maximum salt concentration is a saturated concentration that is an upper limit at which the electrolyte can be dissolved.

Specifically, for example, when the individual electrode 112 and the common electrode 105 are silver/silver chloride electrodes, a potassium chloride aqueous solution having a concentration of 3 M can be used as the droplets 110 and the electrolyte solution 103. The reason is that since chloride ions can undergo an electron transfer reaction with the silver/silver chloride electrodes and potassium ions have the same electrical mobility as the chloride ions, the electrical conductivity can be sufficiently secured. In addition to potassium chloride, other kinds of monovalent cation of an alkali metal may be used as ionic species such as a lithium ion, a sodium ion, a rubidium ion, a cesium ion, or an ammonium ion.

<Conveyance Control of Biopolymer>

When DNA sequencing or RNA sequencing is performed by using the biopolymer analysis equipment 1800, it is necessary to perform conveyance control when DNA or RNA passes through the nanopore. The conveyance control of the biopolymer can be mainly performed by a molecular motor using an enzyme. The conveyance control by the molecular motor needs to be started only in the vicinity of the nanopore. In particular, the start of the conveyance by the molecular motor in the vicinity of the nanopore can be controlled by binding a control chain to the biopolymer to be read. Such a configuration is described in, for example, JP application No. 2018-159481 and International application No. PCT/JP2018/039466. The disclosures of these documents are incorporated as constituting a part of the present specification.

Here, the enzyme used as the molecular motor refers to all enzymes having a binding capacity to the biopolymer. When the biopolymer is DNA, examples of the enzyme include DNA polymerase, DNA helicase, DNA exonuclease, DNA transposase, and the like. When the biopolymer is RNA, examples of the enzyme include RNA polymerase, RNA helicase, RNA exonuclease, RNA transposase, and the like.

As described above, when a voltage is applied to both ends of the nanopore disposed in the electrolyte solution, an electric field is generated in the vicinity of the nanopore 101, and the biopolymer passes through the nanopore by the force. On the other hand, since the molecular motor is generally larger than a nanopore diameter, the molecular motor cannot pass through the nanopore. In order to realize this limitation, it is desirable that the nanopore diameter is in a range from 0.8 nm, which is a lower limit at which single-stranded DNA or single-stranded RNA can pass, to 3 nm, which is an upper limit at which the enzyme as the molecular motor does not pass. Under this condition, a primer in the control chain approaches the molecular motor staying in the vicinity of the nanopore, and thus, an extension and separation reaction is started. As a result, the biopolymer is pulled up or pulled down from the nanopore by the force when the molecular motor extends and separates a complementary chain, and the biopolymer is analyzed from a change in the ion current acquired at this time.

The configuration in which the monomer sequence information in the biopolymer 1 is acquired based on the electrical signal has been described above. The monomer sequence information of the biopolymer 1 can also be obtained by a method for acquiring a tunnel current by providing an electrode inside the nanopore or a method for detecting a change in transistor characteristics. The monomer sequence information of the biopolymer 1 may be acquired based on an optical signal. That is, a method for deciding each monomer sequence by providing a label having a characteristic fluorescence wavelength for each monomer and measuring the fluorescence signal may be used.

The biopolymer analysis device (nanopore device) for analyzing the biopolymer and the biopolymer analysis equipment including the same include the above-described components as elements. The biopolymer analysis device and the biopolymer analysis equipment can be provided together with instructions describing a use procedure, a use amount, and the like. The biopolymer analysis device may be provided in a state in which the nanopore is formed in an immediately usable state, or may be provided in a state in which the nanopore is formed in a providing destination.

### Modification Example

The present disclosure is not limited to the above-described embodiments, and includes various modification examples. For example, the aforementioned embodiments are described in detail in order to facilitate easy understanding of the present disclosure, and are not limited to necessarily include all the described components. A part of a certain embodiment may be replaced with the configuration of another embodiment. The configuration of another embodiment can be added to the configuration of a certain embodiment. A part of the configuration of another embodiment can be added, deleted, or replaced to, from, or with a part of the configuration of each embodiment.

All publications and patent literatures cited in the present specification are hereby incorporated by reference in the present specification.

### REFERENCE SIGNS LIST

1 biopolymer
101 nanopore
102 thin film
103 electrolyte solution
104A first liquid tank
104B second liquid tank
105 common electrode
106 ammeter
107 power supply
108 computer
110 droplet
111 water-repellent liquid
112 individual electrode
113 substrate
114 EWOD electrode
115 insulator
116 EWOD conveying circuit
117 nanopore opening circuit
118 current measuring circuit
119 substrate
120 opening
121 control circuit
122 switch
123 capacitor

1. A biopolymer analysis device, comprising:
an insulating thin film that is made of an inorganic material;
a first liquid tank and a second liquid tank that are separated by the thin film;
a plurality of first electrodes that is arranged in the first liquid tank; and
a second electrode that is disposed in the second liquid tank,
wherein a water-repellent liquid and a plurality of liquid droplets are introduced into the first liquid tank,
the plurality of first electrodes is configured to be able to convey the plurality of droplets introduced into the first liquid tank by electro wetting on dielectric by applying a certain voltage, and
the plurality of droplets is conveyed to portions coming into contact with the plurality of first electrodes, and is insulated from each other by the water-repellent liquid.

2. The biopolymer analysis device according to claim 1, wherein
the first liquid tank further includes a plurality of third electrodes,
the plurality of droplets is conveyed to portions coming into contact with the plurality of first electrodes and the plurality of third electrodes, respectively, and
the plurality of third electrodes is configured to be able to measure a current flowing to the second liquid tank from each of the plurality of droplets via the thin film.

3. The biopolymer analysis device according to claim 1, wherein the plurality of first electrodes includes insulating films on surfaces.

4. The biopolymer analysis device according to claim 1, wherein the plurality of first electrodes is configured to be able to further measure a current flowing to the second liquid tank from each of the plurality of droplets via the thin film.

5. The biopolymer analysis device according to claim 2, wherein a nanopore is formed in the thin film by applying a dielectric breakdown voltage of the thin film between the plurality of third electrodes and the second electrode.

6. The biopolymer analysis device according to claim 4, wherein a nanopore is formed in the thin film by applying a dielectric breakdown voltage of the thin film between the plurality of first electrodes and the second electrode.

7. The biopolymer analysis device according to claim 2, wherein the plurality of first electrodes is arranged around the plurality of third electrodes to form a lane through which the plurality of droplets is conveyed.

8. The biopolymer analysis device according to claim 1, further comprising:
a mechanism for determining whether or not the plurality of droplets is conveyed to desired positions.

9. The biopolymer analysis device according to claim 1, wherein the thin film has a recess of which a cross-sectional shape is a tapered shape at a portion where the droplet is conveyed.

10. The biopolymer analysis device according to claim 2, wherein any one of the plurality of first electrodes and the plurality of third electrodes are provided on the thin film.

11. The biopolymer analysis device according to claim 1, wherein
a plurality of the second electrodes is provided in the second liquid tank, and the water-repellent liquid and the plurality of droplets are introduced into the second liquid tank,
the plurality of second electrodes is configured to be able to convey the plurality of droplets introduced into the second liquid tank by the electro wetting on dielectric by applying the certain voltage, and
the plurality of droplets is conveyed to portions coming into contact with the plurality of second electrodes, and is insulated from each other by the water-repellent liquid.

12. Biopolymer analysis equipment, comprising:
the biopolymer analysis device according to claim 1; and
a controller that controls a voltage applied to the plurality of first electrodes and the second electrode,
wherein the controller includes
an EWOD voltage applying circuit that applies the certain voltage to the plurality of first electrodes,
a nanopore opening circuit that forms a nanopore by applying a dielectric breakdown voltage of the thin film between the plurality of first electrodes and the second electrode,
a current measuring circuit that measures a current flowing between the plurality of first electrodes and the second electrode, and
switches that switch between connections of the EWOD voltage applying circuit, the nanopore opening circuit, or the current measuring circuit, and the plurality of first electrodes.

13. The biopolymer analysis equipment according to claim 12, wherein insulators are arranged between the EWOD voltage applying circuit and the plurality of first electrodes.

14. A biopolymer analysis method, comprising:
preparing a biopolymer analysis device that includes an insulating thin film made of an inorganic material, a first liquid tank and a second liquid tank separated by the thin film, a plurality of first electrodes arranged in the first liquid tank, and a second electrode disposed in the second liquid tank, the plurality of first electrodes being configured to be able to convey a plurality of droplets introduced into the first liquid tank by electro wetting on dielectric by applying a certain voltage;
introducing a water-repellent liquid into the first liquid tank;
introducing the plurality of droplets into the first liquid tank;
conveying the plurality of droplets to portions coming into contact with the plurality of first electrodes by applying the certain voltage to the plurality of first electrodes and insulating the plurality of droplets from each other by the water-repellent liquid; and
introducing an electrolyte solution into the second liquid tank.

15. The biopolymer analysis method according to claim 14, wherein
the first liquid tank further includes a plurality of third electrodes,
the plurality of droplets is conveyed to portions coming into contact with the plurality of first electrodes and the plurality of third electrodes, respectively, and
the biopolymer analysis method further comprises measuring a current flowing between each of the plurality of third electrodes and the second electrode.

16. The biopolymer analysis method according to claim 15, further comprising:
forming a nanopore in the thin film by applying a dielectric breakdown voltage of the thin film between each of the plurality of third electrodes and the second electrode.

17. The biopolymer analysis method according to claim 14, wherein
the plurality of first electrodes and the second electrode are connected to a controller that controls a voltage applied to the first electrodes and the second electrode, and
the controller includes
an EWOD voltage applying circuit that applies the certain voltage to the plurality of first electrodes,
a nanopore opening circuit that forms a nanopore by applying a dielectric breakdown voltage of the thin film between the plurality of first electrodes and the second electrode,
a current measuring circuit that measures a current flowing to the thin film between the plurality of first electrodes and the second electrode, and
switches that switch between connections of the EWOD voltage applying circuit, the nanopore opening circuit, or the current measuring circuit, and the plurality of first electrodes.

18. The biopolymer analysis method according to claim 14, wherein
the plurality of droplets is droplets containing biopolymers, and
the biopolymer analysis method further comprises
forming a nanopore by applying a dielectric breakdown voltage of the thin film between the plurality of first electrodes and the second electrode,
applying a voltage at which the biopolymer is capable of being electrophoresed between the plurality of first electrodes and the second electrode, and
analyzing the biopolymer based on a current value flowing between the plurality of first electrodes and the second electrode when the biopolymer passes the nanopore.

19. The biopolymer analysis method according to claim 14, further comprising:
determining whether or not the plurality of droplets is conveyed to desired positions.
